(19) United States
(12) Patent Application Publication
(10) Pub. No.: US 2023/0372727 A1
(43) Pub. Date: Nov. 23, 2023

(54) ENDOSCOPE CAP, LIGHT TREATMENT ENDOSCOPE SYSTEM, AND LIGHT TREATMENT METHOD

(71) Applicant: OLYMPUS CORPORATION, Tokyo (JP)

(72) Inventor: Masaru MIZUNAKA, Tokyo (JP)

(73) Assignee: OLYMPUS CORPORATION, Tokyo (JP)

(21) Appl. No.: 18/200,207

(22) Filed: May 22, 2023

Related U.S. Application Data
(60) Provisional application No. 63/344,750, filed on May 23, 2022.

(51) Int. Cl. A61N 5/06

(52) U.S. Cl. CPC A61N 5/0603; A61N 2005/0663

(57) ABSTRACT

An endoscope cap includes: a light-transmissive portion having a tubular shape made of a light-transmissive material, the light-transmissive portion including a coating layer on a periphery of the tubular shape; and a tubular portion configured to connect the light-transmissive portion to a distal end of an insertion portion of an endoscope, the coating layer being configured to reflect light in a wavelength band for light treatment of a body portion and transmit light in a wavelength band for white light image capturing.

### CROSS REFERENCES TO RELATED APPLICATIONS

This application is based on and claims priority under 35 U.S.C. § 119 to U.S. Provisional Application No. 63/344,750, filed on May 23, 2022, the entire contents of which is incorporated herein by reference.

### BACKGROUND

The present disclosure relates to an endoscope cap, a light treatment endoscope system, and a light treatment method.

In the related art, studies have been conducted on photo-immuno therapy (PIT) in which an antibody agent including an antibody that binds to the surface of a cancer cell and a phthalocyanine derivative IR700 is administered to a subject, and is irradiated with light in a wavelength band for light treatment to specifically kill only the cancer cell (see, for example, JP 2020-72969 A and JP 2020-114467 A). At this time, the antibody agent emits fluorescence by being excited by the irradiation with the light in the wavelength band for light treatment. Then, the fluorescence intensity is used as an index of a treatment effect. Therefore, by capturing the fluorescence with an imaging device, it is possible to grasp the treatment effect from the fluorescence intensity captured by the imaging device.

### SUMMARY

In some embodiments, an endoscope cap includes: a light-transmissive portion having a tubular shape made of a light-transmissive material, the light-transmissive portion including a coating layer on a periphery of the tubular shape; and a tubular portion configured to connect the light-transmissive portion to a distal end of an insertion portion of an endoscope, the coating layer being configured to reflect light in a wavelength band for light treatment of a body portion and transmit light in a wavelength band for white light image capturing.

In some embodiments, a light treatment endoscope system includes: a first light source configured to supply a first light in a first wavelength band for light treatment; a second light source configured to supply a second light in a second wavelength band for white light imaging; an endoscope including an insertion portion, the endoscope being configured to emit the first light and the second light from a distal end of the insertion portion; and an endoscope cap detachably connected to the distal end of the insertion portion, the endoscope cap including a light-transmissive portion and a tubular portion, the light-transmissive portion having a tubular shape made of a light-transmissive material and including a coating layer on a periphery of the tubular shape, the tubular portion being configured to connect the light-transmissive portion to the distal end of the insertion portion, the light-transmissive portion being configured to be fixed to the distal end of the insertion portion to surround an emission site at the distal end of the insertion portion when viewed from a direction along a central axis of the insertion portion, the first light and the second light are emitted from the emission site, the coating layer being configured to reflect the first light and transmit the second light.

In some embodiments, a light treatment method includes: fixing an endoscope cap having a tubular shape to a distal end of an insertion portion of an endoscope such that the tubular shape surrounds an emission site at the distal end of the insertion portion when viewed from a direction along a central axis of the insertion portion, the endoscope cap including a coating layer on a periphery of the tubular shape, the coating layer being configured to reflect first light in a first wavelength band for light treatment and transmit second light in a second wavelength band for white light imaging; pressing a distal end of the endoscope cap against a living tissue; concurrently with the pressing, emitting the second light to acquire a white light image of the living tissue based on the second light; and concurrently with the pressing, irradiating a treatment target of the living tissue with the first light to treat the living tissue.

### BRIEF DESCRIPTION OF THE DRAWINGS

FIG. 1 is a diagram illustrating a configuration of an endoscope system according to an embodiment;

FIG. 2 is a diagram illustrating a configuration of an endoscope cap;

FIG. 3 is a diagram illustrating transmission characteristics of a coating layer;

FIG. 4 is a flowchart illustrating a light treatment method;

FIG. 5 is a diagram describing a light treatment method;

FIG. 6 is a diagram describing a light treatment method;

FIG. 7 is a diagram describing a light treatment method;

FIG. 8 is a diagram describing a light treatment method;

FIG. 9 is a diagram describing a light treatment method;

FIG. 10 is a diagram describing a light treatment method;

FIG. 11 is a diagram describing a light treatment method;

FIG. 12 is a diagram describing a first modification of the embodiment;

FIG. 13 is a diagram describing a second modification of the embodiment; and

FIG. 14 is a diagram describing a third modification of the embodiment.

### DETAILED DESCRIPTION

Hereinafter, a mode for carrying out the disclosure (hereinafter, the embodiment) will be described with reference to the drawings. Note that the disclosure is not limited by the embodiment described below. Further, in the description of the drawings, the same portions are denoted by the same reference numerals.

Configuration of Endoscope System

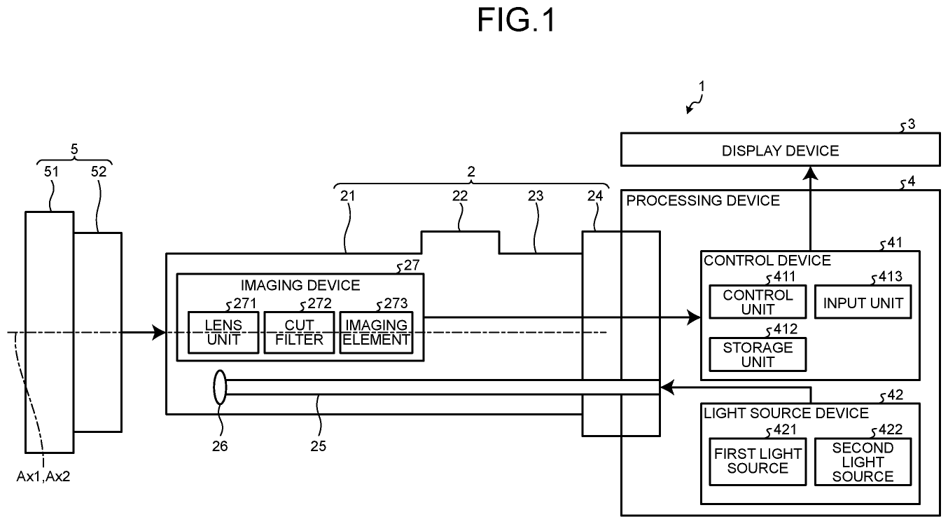

An endoscope system 1 is a system that is used in the medical field and performs treatment while observing the inside of a subject (inside of a living body). As illustrated in FIG. 1, the endoscope system 1 includes an endoscope 2, a display (hereinafter display device) 3, a processor (hereinafter processing device) 4, and an endoscope cap 5.

In the present embodiment, the endoscope 2 is a so-called flexible endoscope. The endoscope 2 is partially inserted into a living body, captures the inside of the living body, and outputs an image signal generated by the capturing. Then, as illustrated in FIG. 1, the endoscope 2 includes an insertion portion 21, an operating unit 22, a universal cord 23, and a connector 24.

The insertion portion 21 is a portion at least a part of which has flexibility and is configured to be inserted into a living body. In the insertion portion 21, a light guide 25, an illumination lens 26, and an imaging device 27 are provided.

The light guide 25 is routed from the insertion portion 21 to the connector 24 through the operating unit 22 and the universal cord 23. Then, one end of the light guide 25 is located at a distal end portion in the insertion portion 21. In addition, in a state where the endoscope 2 is connected to the processing device 4, the other end of the light guide 25 is located in the processing device 4. Then, the light guide 25 transmits light supplied from a light source device 42 in the processing device 4 from the other end to the one end.

The illumination lens 26 faces the one end of the light guide 25 in the insertion portion 21. Then, the illumination lens 26 irradiates the inside of the living body with the light transmitted by the light guide 25.

The imaging device 27 is provided at the distal end portion in the insertion portion 21. Then, the imaging device 27 captures the inside of the living body, and outputs an image signal generated by the capturing. As illustrated in FIG. 1, the imaging device 27 includes a lens unit 271, a cut filter 272, and an imaging sensor (hereinafter imaging element) 273.

The lens unit 271 takes a subject image and forms the subject image on a light receiving surface of the imaging element 273.

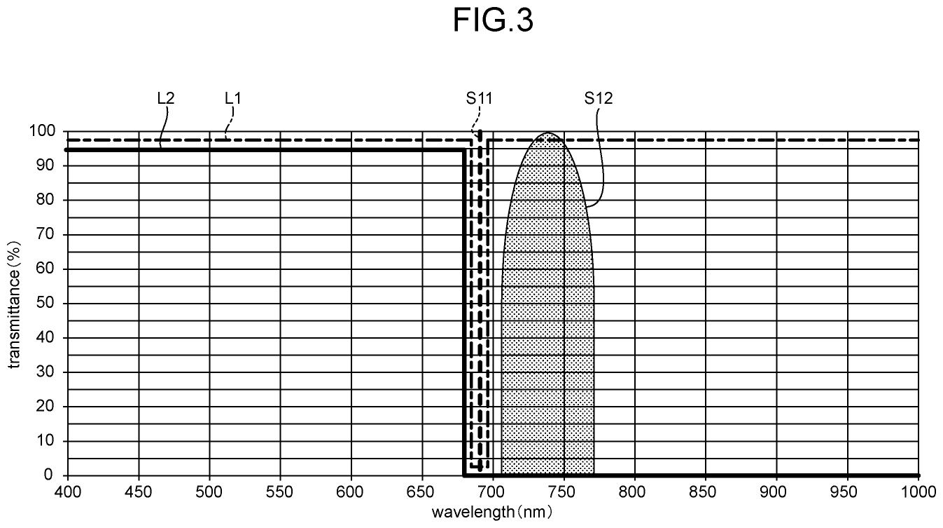

The cut filter 272 is disposed between the lens unit 271 and the imaging element 273, and cuts only light in a second wavelength band to be described below within the light passing through the lens unit 271. In the present embodiment, as will be described below, light in a wavelength band for light treatment (wavelength longer than 680 nm (about 690 nm)) used in the PIT (hereinafter, described as first excitation light) is adopted as the light in the second wavelength band. Therefore, the cut filter 272 cuts only light having a center wavelength: 690 nm (see a curve L1 indicated by the one-dot chain line in FIG. 3). That is, the cut filter 272 cuts substantially entirely the first excitation light.

Note that the arrangement position of the cut filter 272 is not limited to the position between the lens unit 271 and the imaging element 273, but may be other positions, for example, in the lens unit 271 as long as it is the front stage of the optical path of the imaging element 273.

Accordingly, in a case where the inside of the living body is irradiated with only light in a wavelength band for white light image capturing (hereinafter, described as white light), the subject image includes the white light reflected in the living body. In addition, in a case where the inside of the living body is irradiated with only the first excitation light, the subject image does not include the first excitation light reflected in the living body, and includes only fluorescence emitted from the antibody agent by the first excitation light. Further, in a case where the inside of the living body is simultaneously irradiated with the white light and the first excitation light, the subject image does not include the first excitation light reflected in the living body, and includes the white light reflected in the living body and the fluorescence emitted from the antibody agent by the first excitation light.

The imaging element 273 includes a charge coupled device (CCD), a complementary metal oxide semiconductor (CMOS), or the like that receives a subject image and converts the subject image into an electric signal, and generates an image signal by capturing the subject image.

The operating unit 22 is connected to a proximal end portion of the insertion portion 21. Then, the operating unit 22 receives various operations on the endoscope 2.

The universal cord 23 is a cord that extends from the operating unit 22 in a direction different from the extending direction of the insertion portion 21 and in which a signal line that electrically connect the imaging device 27 and a control device 41 in the processing device 4, the light guide 25, and the like are arranged.

The connector 24 is provided at an end portion of the universal cord 23 and is detachably connected to the processing device 4.

The display device 3 is a liquid crystal display (LCD), an electro luminescence (EL) display, or the like, and displays an image or the like after image processing is executed by the processing device 4.

As illustrated in FIG. 1, the processing device 4 includes the control device 41 and the light source device 42. Note that, in the present embodiment, the light source device 42 and the control device 41 are provided in one casing as the processing device 4, but the present embodiment is not limited thereto, and the light source device 42 and the control device 41 may be provided in different casings.

The light source device 42 supplies specific light to the other end of the light guide 25 under the control of the control device 41. As illustrated in FIG. 1, the light source device 42 includes a first light source 421 and a second light source 422.

The first light source 421 emits light in a first wavelength band. In the present embodiment, the first light source 421 emits white light as the light in the first wavelength band. Examples of the first light source 421 include a light emitting diode (LED) and the like.

The second light source 422 emits the light in the second wavelength band different from the first wavelength band. In the present embodiment, the second light source 422 emits the first excitation light (wavelength longer than 680 nm (about 690 nm)) used in the PIT as the light in the second wavelength band. In addition, when excited by the first excitation light, the antibody agent emits fluorescence having a central wavelength on a long wavelength side with respect to a central wavelength of the wavelength band of the first excitation light. As the second light source 422, a semiconductor laser or the like can be exemplified.

The control device 41 integrally controls the entire operation of the endoscope system 1. As illustrated in FIG. 1, the control device 41 includes a control unit 411, a storage unit 412, and an input unit 413.

The control unit 411 includes a controller such as a central processing unit (CPU) or a micro processing unit (MPU), or an integrated circuit such as an application specific integrated circuit (ASIC) or a field programmable gate array (FPGA), and controls the entire operation of the endoscope system 1.

For example, the control unit 411 acquires an image signal from the imaging element 273 and executes the image processing on the image signal. Examples of the image processing include optical black subtraction processing, white balance adjustment processing, demosaic processing, color correction processing, gamma correction processing, and YC processing for converting RGB signals into luminance signals and color difference signals (Y, C_{B}/C_{R} signal). Then, the control unit 411 causes the display device 3 to display an image based on the image signal after the execution of the image processing. Hereinafter, for convenience of description, an image obtained by performing the above-described image processing on an image signal generated by irradiating the inside of the living body with only the white light and capturing a subject image including the white light will be referred to as a white light image. In addition, an image obtained by performing the above-described image processing on an image signal generated by irradiating the inside of the living body with only the first excitation light and capturing a subject image including fluorescence emitted from an antibody agent by the first excitation light is referred to as a fluorescence image.

The storage unit (memory) 412 stores various programs executed by the control unit 411, information necessary for processing of the control unit 411, and the like.

The input unit 413 includes a keyboard, a mouse, a switch, a touch panel, and the like, and receives a user operation by a user such as an operator. Then, the input unit 413 outputs an operation signal corresponding to the user operation to the control unit 411.

The endoscope cap 5 is a member detachably connected to the distal end of the insertion portion 21.

Hereinafter, the endoscope cap 5 will be described in detail.

Configuration of Endoscope Cap

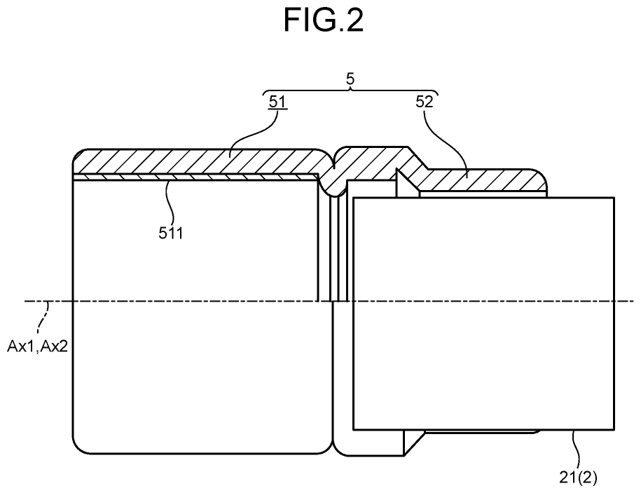

FIG. 2 is a diagram illustrating a configuration of the endoscope cap 5. Note that, in FIG. 2, only a part is illustrated by a cross section for convenience of description.

As illustrated in FIG. 2, the endoscope cap 5 integrally includes a light-transmissive portion 51 and a fixing portion 52. The endoscope cap 5 is made of a light-transmissive resin material.

The light-transmissive portion 51 has a cylindrical shape. In the present embodiment, a distal end of the light-transmissive portion 51 is parallel to a plane orthogonal to a central axis Ax1 (FIG. 2) of the light-transmissive portion 51. In addition, as illustrated in FIG. 2, a coating layer 511 is provided on an inner surface of the light-transmissive portion 51. The position where the coating layer 511 is provided is not limited to the inner surface of the light-transmissive portion 51, but may be an outer surface.

Note that the transmission characteristics of the coating layer 511 will be described below in “Transmission Characteristics of Coating Layer”.

The fixing portion 52 has a cylindrical shape, and is integrally formed at one end of the light-transmissive portion 51 in a state of being coaxial with the light-transmissive portion 51. Then, the fixing portion 52 fixes the light-transmissive portion 51 to the distal end of the insertion portion 21 when the distal end of the insertion portion 21 is fitted. In this state, when viewed from the direction along a central axis Ax1 (FIG. 1) of the insertion portion 21, an emission site (illumination lens 26) that is at the distal end of the insertion portion 21 and which from the first excitation light and the white light are emitted is surrounded by the light-transmissive portion 51.

Transmission Characteristics of Coating Layer

FIG. 3 is a diagram illustrating transmission characteristics of the coating layer 511. Specifically, in FIG. 3, the vertical axis represents transmittance [%], and the horizontal axis represents wavelength [nm]. In addition, in FIG. 3, the curve L1 indicated by the one-dot chain line indicates transmission characteristics of the cut filter 272. In addition, a curve L2 indicated by the solid line indicates transmission characteristics of the coating layer 511. Further, a spectrum S11 indicates a spectrum of the first excitation light. In addition, a spectrum S12 indicates a spectrum of the fluorescence emitted from the antibody agent by the first excitation light.

In the present embodiment, the first excitation light is light in a wavelength band of about 690 nm as indicated by the spectrum S11 in FIG. 3. In addition, the fluorescence emitted from the antibody agent by the first excitation light is light in a wavelength band of about 700 nm as indicated by the spectrum S12 in FIG. 3. Further, although specific illustration is omitted, the white light is light in a wavelength band of less than 680 nm.

Then, as indicated by the curve L2 in FIG. 3, the coating layer 511 reflects the light in the wavelength band of 680 nm or more and transmits the light in the wavelength band of less than 680 nm. That is, the coating layer 511 reflects the first excitation light and the fluorescence emitted from the antibody agent by the first excitation light, and transmits the white light.

Light Treatment Method

Next, a light treatment method will be described.

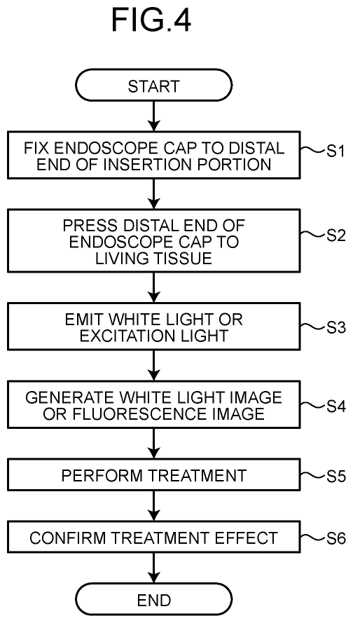

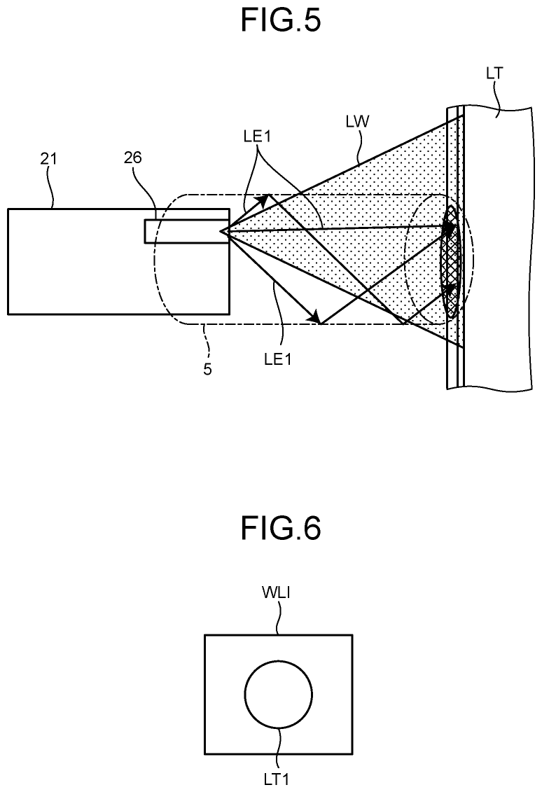

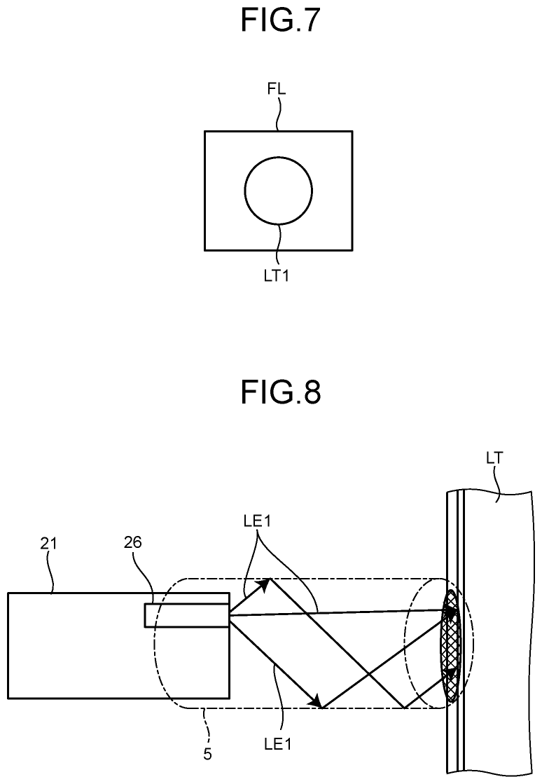

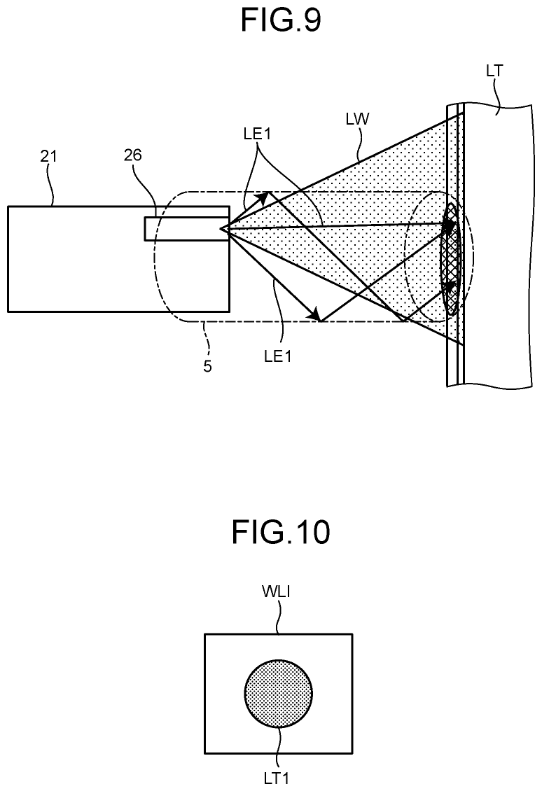

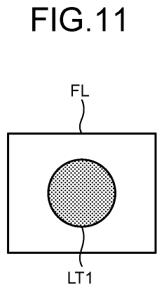

FIG. 4 is a flowchart illustrating a light treatment method. FIGS. 5 to 11 are diagrams describing the light treatment method. Specifically, FIG. 5 is a diagram illustrating Steps S1 to S3, and is a diagram illustrating a positional relationship between the endoscope cap 5 attached to the distal end of the insertion portion 21 and a living tissue LT. FIG. 6 is a diagram illustrating a white light image WLI generated in Step S4. FIG. 7 is a diagram illustrating a fluorescence image FL generated in Step S4. FIG. 8 is a diagram corresponding to FIG. 5 and is a diagram illustrating Step S5. FIG. 9 is a diagram corresponding to FIG. 5 and is a diagram illustrating Step S6. FIG. 10 is a diagram illustrating a white light image WLI generated in Step S6. FIG. 11 is a diagram illustrating a fluorescence image FL generated in Step S6.

First, as illustrated in FIG. 5, the user such as the operator fixes the endoscope cap 5 to the distal end of the insertion portion 21 (Step S1).

After Step S1, the user such as the operator presses the distal end of the endoscope cap 5 against the living tissue LT as illustrated in FIG. 5 (Step S2).

After Step S2, the user such as the operator operates the operating unit 22 or the input unit 413. As a result, the control unit 411 operates the first light source 421 or the second light source 422 to emit white light LW or first excitation light LE1 as illustrated in FIG. 5 (Step S3).

Here, as illustrated in FIG. 5, the white light LW emitted from the distal end (illumination lens 26) of the insertion portion 21 is emitted to the living tissue LT without being reflected by the coating layer 511 formed on the inner surface of the endoscope cap 5, that is, without limitation of the irradiation region by the endoscope cap 5. In addition, the white light LW reflected from the living tissue LT is also taken into the imaging device 27 without limitation by the endoscope cap 5.

On the other hand, as illustrated in FIG. 5, substantially all of the first excitation light LE1 emitted from the distal end (illumination lens 26) of the insertion portion 21 is reflected by the coating layer 511 formed on the inner surface of the endoscope cap 5, and is emitted to a site of the living tissue LT located in the endoscope cap 5. In addition, substantially all of the first excitation light LE1 reflected at the site and the fluorescence from the site are taken into the imaging device 27 while being reflected by the coating layer 511.

After Step S3, the control unit 411 acquires an image signal from the imaging element 273 and executes the image processing on the image signal. As a result, the control unit 411 generates the white light image WLI illustrated in FIG. 6 or the fluorescence image FL illustrated in FIG. 7 (Step S4). Then, the control unit 411 causes the display device 3 to display the generated white light image WLI or fluorescence image FL.

Note that, in Step S4, in a case where both the white light image WLI and the fluorescence image FL are generated, the control unit 411 may generate a superimposed image in which the white light image WLI and the fluorescence image FL are superimposed between corresponding pixels, and may cause the display device 3 to display the superimposed image.

Here, the user such as the operator moves the distal end of the insertion portion 21 in a state where the distal end of the endoscope cap 5 is pressed against the living tissue LT while confirming the white light image WLI or the fluorescence image FL displayed on the display device 3, and searches for a treatment target LT1 (FIGS. 6 and 7). As the treatment target LT1, a tumor can be exemplified. Note that the antibody agent has already been administered to the treatment target LT1. The administration of the antibody agent may be performed using the endoscope 2, may be performed using another equipment, or may be performed by causing a patient to take the agent.

After Step S4, the user such as the operator operates the operating unit 22 or the input unit 413. As a result, the control unit 411 operates only the second light source 422 of the first and second light sources 421 and 422 to emit the first excitation light LE1 as illustrated in FIG. 8. That is, the treatment is performed on the treatment target LT1 (Step S5).

Note that the first excitation light LE1 emitted from the second light source 422 is used for treatment, but it is not limited thereto. For example, the first excitation light LE1 is used to confirm a treatment effect. Then, the light in the second wavelength band similar to the first excitation light LE1 may be emitted from another light source different from the second light source 422, and the light may be used as treatment light used for treatment. For example, it is possible to adopt a configuration in which the treatment target LT1 is irradiated with the treatment light from a treatment tool inserted into a treatment tool channel (illustration omitted) provided in the insertion portion 21 and protruding from the distal end of the insertion portion 21.

In addition, during the treatment of the treatment target LT1 in Step S5, the control unit 411 may generate one of the fluorescence image FL and the superimposed image in which the white light image WLI and the fluorescence image FL are superimposed between corresponding pixels, and may cause the display device 3 to display the one image.

After Step S5, the user such as the operator operates the operating unit 22 or the input unit 413. As a result, the control unit 411 operates the first light source 421 or the second light source 422 to emit the white light LW or the first excitation light LE1 as illustrated in FIG. 9. In addition, the control unit 411 acquires an image signal from the imaging element 273 and executes the image processing on the image signal. As a result, the control unit 411 generates the white light image WLI illustrated in FIG. 10 or the fluorescence image FL illustrated in FIG. 11. Then, the control unit 411 causes the display device 3 to display the generated white light image WLI or fluorescence image FL. The user such as the operator confirms the treatment effect of the treatment target LT1 by confirming the white light image WLI or the fluorescence image FL displayed on the display device 3 (Step S6).

Note that, in Step S6, in a case where both the white light image WLI and the fluorescence image FL are generated, the control unit 411 may generate a superimposed image in which the white light image WLI and the fluorescence image FL are superimposed between corresponding pixels, and may cause the display device 3 to display the superimposed image.

According to the present embodiment described above, the effects described below are obtained.

The endoscope cap 5 according to the present embodiment includes the light-transmissive portion 51 in which the coating layer 511 is provided on the inner surface having a tubular shape, and the fixing portion 52 that fixes the light-transmissive portion 51 to the distal end of the insertion portion 21. Then, the coating layer 511 has transmission characteristics of reflecting the first excitation light LE1. That is, in a state where the endoscope cap 5 is pressed against the living tissue LT, substantially all of the first excitation light LE1 emitted from the distal end of the insertion portion 21 is reflected by the coating layer 511, and is emitted to a site of the living tissue LT located in the endoscope cap 5. Therefore, even when the output of the first excitation light LE1 from the second light source 422 is not increased, a specific region can be efficiently irradiated with the first excitation light LE1.

Accordingly, with the endoscope cap 5 according to the present embodiment, it is possible to uniformly emit the desired light amount of the first excitation light LE1 in a stable state while suppressing the output of the second light source 422 that emits the first excitation light LE1. In addition, downsizing and cost reduction of the second light source 422 can be achieved.

Since the first excitation light LE1 is emitted from the distal end of the insertion portion 21 in a state where the endoscope cap 5 is pressed against the living tissue LT, the distance between the living tissue LT and the distal end of the insertion portion 21 does not change. Therefore, the irradiation range and the irradiation density of the first excitation light LE1 can be kept constant, and the treatment of the treatment target LT1 can be stably performed.

In addition, with the endoscope cap 5 according to the present embodiment, the coating layer 511 has transmission characteristics of transmitting the white light LW. Therefore, the visual field of the white light LW is not limited by the endoscope cap 5. In addition, the white balance of the white light image WLI is not lost by the endoscope cap 5.

In addition, with the endoscope cap 5 according to the present embodiment, the coating layer 511 has transmission characteristics of reflecting the fluorescence emitted from the antibody agent by the first excitation light LE1. That is, substantially all of the fluorescence emitted from the antibody agent present at a site of the living tissue LT by the first excitation light LE1 emitted to the site located in the endoscope cap 5, the first excitation light LE1 being emitted from the distal end of the insertion portion 21 in a state where the endoscope cap 5 is pressed against the living tissue LT, is reflected by the coating layer 511 and is taken into the imaging device 27. Therefore, the fluorescence can be efficiently taken in, the fluorescence intensity of the fluorescence on the fluorescence image FL can be further increased, and the treatment effect can be favorably confirmed.

### Other Embodiments

Although an exemplary embodiment for carrying out the disclosure has been described so far, the disclosure should not be limited only by the above-described embodiment.

For example, in the above-described embodiment, a configuration of first to third modifications described below may be adopted.

Hereinafter, the first to third modifications will be sequentially described.

First Modification

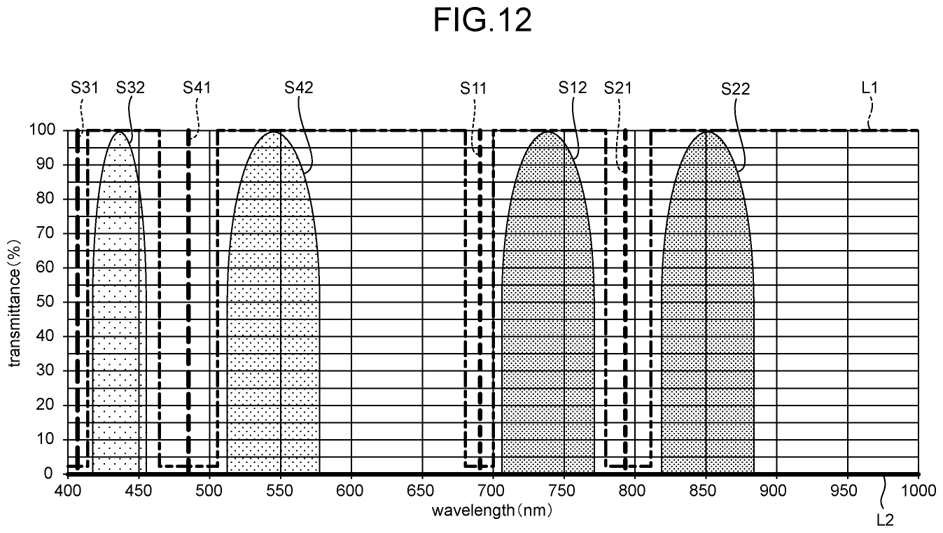

FIG. 12 is a diagram describing the first modification of the embodiment. Specifically, FIG. 12 is a diagram corresponding to FIG. 3. In FIG. 12, a curve L1 indicated by the one-dot chain line indicates transmission characteristics of a cut filter 272 according to the present first modification. In addition, a curve L2 indicated by the solid line indicates transmission characteristics of a coating layer 511 according to the present first modification. Further, a spectrum S21 indicates a spectrum of second excitation light according to the present first modification. In addition, a spectrum S22 indicates a spectrum of the fluorescence emitted from the antibody agent by the second excitation light. Further, a spectrum S31 indicates a spectrum of third excitation light according to the present first modification. In addition, a spectrum S32 indicates a spectrum of the fluorescence emitted from the antibody agent by the third excitation light. Further, a spectrum S41 indicates a spectrum of fourth excitation light according to the present first modification. In addition, a spectrum S42 indicates a spectrum of the fluorescence emitted from the antibody agent by the fourth excitation light.

In the embodiment described above, the first excitation light LE1 was the light in the wavelength band of about 690 nm as indicated by the spectrum S11 in FIG. 12. In addition, the fluorescence emitted from the antibody agent by the first excitation light LE1 was the light in the wavelength band of about 700 nm as indicated by the spectrum S12 in FIG. 12.

However, the excitation light is not limited to the first excitation light LE1, and the second to fourth excitation light according to the present first modification may be adopted.

The second excitation light is light in a wavelength band of about 780 nm as indicated by the spectrum S21 in FIG. 12. In addition, the fluorescence emitted from the antibody agent by the second excitation light is light in a wavelength band of about 800 nm as indicated by the spectrum S22 in FIG. 12.

The third excitation light is light in a wavelength band of about 400 nm as indicated by the spectrum S31 in FIG. 12. In addition, the fluorescence emitted from the antibody agent by the third excitation light is light in a wavelength band of about 420 to 460 nm as indicated by the spectrum S32 in FIG. 12.

The fourth excitation light is light in a wavelength band of about 488 nm as indicated by the spectrum S41 in FIG. 12. In addition, the fluorescence emitted from the antibody agent by the fourth excitation light is light in a wavelength band of about 520 nm as indicated by the spectrum S42 in FIG. 12.

Here, the cut filter 272 according to the present first modification has transmission characteristics of cutting the light of the first excitation light LE1 and the second to fourth excitation light and transmitting the light (fluorescence indicated by the spectra S12, S22, S32, and S42) in other wavelength bands as indicated by the curve L1 indicated by the one-dot chain line in FIG. 12.

In addition, the coating layer 511 according to the present first modification has transmission characteristics of reflecting the light in all the wavelength bands as indicated by the curve L2 indicated by the solid line in FIG. 12. That is, substantially all of the white light LW, the first excitation light LE1, and the second to fourth excitation light emitted from the distal end (illumination lens 26) of the insertion portion 21 are reflected by the coating layer 511 formed on the inner surface of the endoscope cap 5, and are emitted to a site of the living tissue LT located in the endoscope cap 5. In addition, substantially all of the white light LW, the first excitation light LE1, and the second to fourth excitation light reflected at the site, and each fluorescence from the site are taken into the imaging device 27 while being reflected by the coating layer 511.

Even in a case where the configuration according to the present first modification described above is adopted, the same effects as those of the above-described embodiment are obtained.

Second Modification

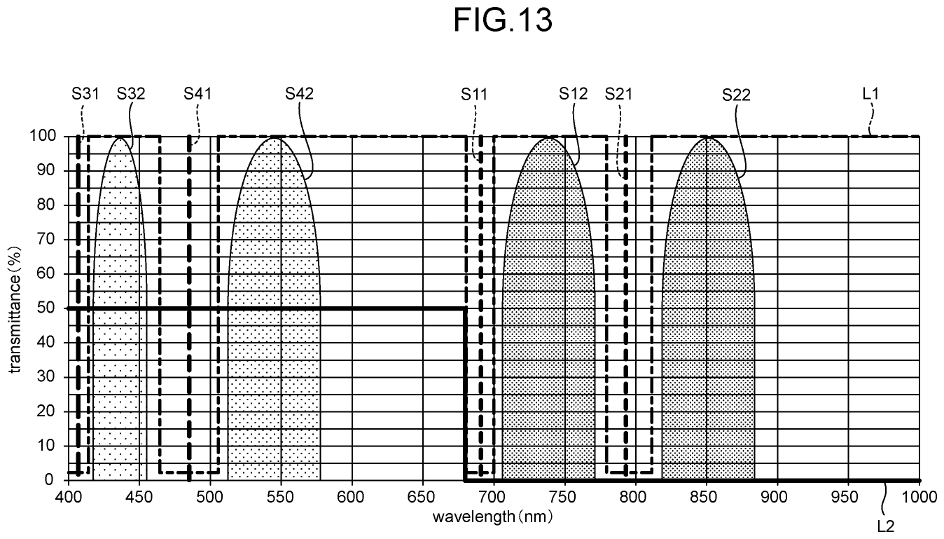

FIG. 13 is a diagram describing the second modification of the embodiment. Specifically, FIG. 13 is a diagram corresponding to FIG. 12. In FIG. 13, a curve L2 indicated by the solid line indicates transmission characteristics of a coating layer 511 according to the present second modification.

In the first modification described above, the transmission characteristics of a curve L2 illustrated in FIG. 13 may be adopted as the transmission characteristics of the coating layer 511.

Specifically, as indicated by the curve L2 in FIG. 13, the coating layer 511 reflects the light in the wavelength band of 680 nm or more. In addition, the coating layer 511 has a transmittance of 50% for a wavelength band of less than 680 nm.

That is, substantially all of the first excitation light LE1 and the second excitation light emitted from the distal end (illumination lens 26) of the insertion portion 21 are reflected by the coating layer 511 formed on the inner surface of the endoscope cap 5, and are emitted to a site of the living tissue LT located in the endoscope cap 5. In addition, substantially all of the first excitation light LE1 and the second excitation light reflected at the site and each fluorescence from the site are taken into the imaging device 27 while being reflected by the coating layer 511.

On the other hand, light of approximately half the light amount of the white light LW, light of approximately half the light amount of the third excitation light, and light of approximately half the light amount of the fourth excitation light emitted from the distal end (illumination lens 26) of the insertion portion 21 are emitted to the living tissue LT without being reflected by the coating layer 511, that is, without limitation of the irradiation region by the endoscope cap 5. In addition, the remaining approximately half of the light amount of the white light LW, the remaining approximately half of the light amount of the third excitation light, and the remaining approximately half of the light amount of the fourth excitation light emitted from the distal end (illumination lens 26) of the insertion portion 21 are reflected by the coating layer 511, and emitted to a site of the living tissue LT located in the endoscope cap 5. Regarding the white light LW and the third and fourth excitation light reflected by the living tissue LT and the fluorescence generated by the third and fourth excitation light, only light of approximately half the light amount is reflected by the coating layer 511.

Even in a case where the configuration according to the present second modification described above is adopted, the same effects as those of the above-described embodiment and first modification are obtained.

Third Modification

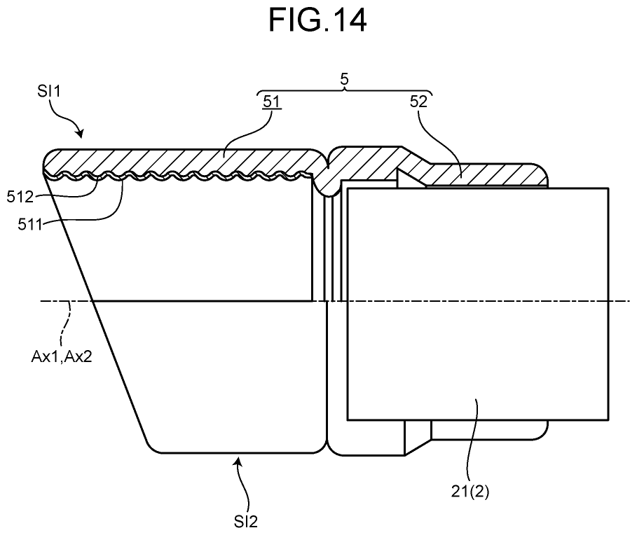

FIG. 14 is a diagram describing the third modification of the embodiment. Specifically, FIG. 14 is a diagram corresponding to FIG. 2.

In the above-described embodiment, the distal end of the endoscope cap 5 (light-transmissive portion 51) may be parallel to a plane inclined with respect to a plane orthogonal to the central axis Ax1 of the light-transmissive portion 51 as in the present third modification illustrated in FIG. 14. That is, the distal end may have a shape inclined in a state of intersecting the central axis Ax1.

Hereinafter, for convenience of description, in the endoscope cap 5 according to the present third modification, a long side in a direction along the central axis Ax1 is referred to as a long side SI1 (FIG. 14), and a short side is referred to as a short side SI2 (FIG. 14).

Then, in the endoscope cap 5 according to the present third modification, as illustrated in FIG. 14, a diffusion portion 512 that diffuses incident light is provided on the long side SI1 on the inner surface of the light-transmissive portion 51. In the present third modification, the diffusion portion 512 is provided on the inner surface of the light-transmissive portion 51 over substantially a half the circumference in a circumferential direction about the central axis Ax1. In addition, the diffusion portion 512 is formed on the inner surface of the light-transmissive portion 51 by surface processing or the like. The position where the diffusion portion 512 is provided is not limited to the inner surface of the light-transmissive portion 51, but may be an outer surface.

According to the present third modification described above, the effects described below are obtained in addition to the effects similar to those described in the above-described embodiment.

In the present third modification, since the distal end of the light-transmissive portion 51 is inclined in a state of intersecting the central axis Ax1, it is possible to treat the living tissue LT positioned in a specific posture with respect to the insertion direction of the insertion portion 21.

Meanwhile, in a case where the distal end of the light-transmissive portion 51 has an inclined shape in a state of intersecting the central axis Ax1 as in the endoscope cap 5 according to the present third modification, the problem described below is likely to occur.

That is, when the living tissue LT is irradiated with the first excitation light LE1, the irradiation intensity of the first excitation light LE1 becomes weak at a site of the living tissue LT located on the long side SI1 within the site located in the endoscope cap 5, and the irradiation intensity on the long side SI1 and the short side SI2 becomes non-uniform.

On the other hand, in the endoscope cap 5 according to the present third modification, the diffusion portion 512 is provided on the long side SI1 of the inner surface of the light-transmissive portion 51. Therefore, by the diffusion portion 512, the irradiation intensity of the first excitation light LE1 with respect to a site located on the long side SI1 and the irradiation intensity of the first excitation light LE1 with respect to a site located on the short side S12 within the site of the living tissue LT located in the endoscope cap 5 can be made uniform.

With the endoscope cap, the light treatment endoscope system, and the light treatment method according to the disclosure, it is possible to uniformly irradiate with light in a state where a desired light amount of a wavelength band for light treatment is stabilized while suppressing an output of a light source that emits light in the wavelength band for light treatment.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope cap comprising:
a light-transmissive portion having a tubular shape made of a light-transmissive material, the light-transmissive portion including a coating layer on a periphery of the tubular shape; and
a tubular portion configured to connect the light-transmissive portion to a distal end of an insertion portion of an endoscope,
the coating layer being configured to reflect light in a wavelength band for light treatment of a body portion and transmit light in a wavelength band for white light image capturing.

2. The endoscope cap according to claim 1, wherein
the coating layer is configured to reflect light in a wavelength band of 680 nm or more and transmit light in a wavelength band of less than 680 nm.

3. The endoscope cap according to claim 1, wherein
the coating layer is provided on an inner surface of the light-transmissive portion.

4. The endoscope cap according to claim 1, wherein
a distal end of the light-transmissive portion is inclined to intersect a central axis of the light-transmissive portion.

5. The endoscope cap according to claim 4, wherein the coating layer is configured to diffuse light, the coating layer being provided circumferentially on less than an entire circumferential periphery of the light-transmissive portion including on a long side of the light-transmissive portion.

6. The endoscope cap according to claim 1, wherein
the coating layer is configured such that a treatment target is irradiated with the light in the wavelength band for light treatment, and the coating layer is configured to reflect fluorescence from the treatment target excited by the light in the wavelength band for light treatment.

7. The endoscope cap according to claim 5, wherein
the coating layer being half of the entire circumferential periphery.

8. A light treatment endoscope system comprising:
a first light source configured to supply a first light in a first wavelength band for light treatment;
a second light source configured to supply a second light in a second wavelength band for white light imaging;
an endoscope including an insertion portion, the endoscope being configured to emit the first light and the second light from a distal end of the insertion portion; and
an endoscope cap detachably connected to the distal end of the insertion portion,
the endoscope cap including a light-transmissive portion and a tubular portion,
the light-transmissive portion having a tubular shape made of a light-transmissive material and including a coating layer on a periphery of the tubular shape,
the tubular portion being configured to connect the light-transmissive portion to the distal end of the insertion portion,
the coating layer being configured to reflect the first light and transmit the second light.

9. The light treatment endoscope system according to claim 8, wherein
the coating layer is configured to reflect light in a wavelength band of 680 nm or more and transmit light in a wavelength band of less than 680 nm.

10. The light treatment endoscope system according to claim 8, wherein
the coating layer is provided on an inner surface of the light-transmissive portion.

11. The light treatment endoscope system according to claim 8, wherein
a distal end of the light-transmissive portion is inclined to intersect a central axis of the light-transmissive portion.

12. The light treatment endoscope system according to claim 8, wherein the coating layer is configured to diffuse light, the coating layer being provided circumferentially on less than an entire circumferential periphery of the light-transmissive portion including on a long side of the light-transmissive portion.

13. The light treatment endoscope system according to claim 8, wherein
the coating layer is configured such that a treatment target is irradiated with the light in the wavelength band for light treatment, and the coating layer is configured to reflect fluorescence from the treatment target excited by the light in the wavelength band for light treatment.

14. The light treatment endoscope system according to claim 12, wherein
the coating layer being half of the entire circumferential periphery.

15. A light treatment method comprising:
fixing an endoscope cap having a tubular shape to a distal end of an insertion portion of an endoscope such that the tubular shape surrounds an emission site at the distal end of the insertion portion when viewed from a direction along a central axis of the insertion portion, the endoscope cap including a coating layer on a periphery of the tubular shape, the coating layer being configured to reflect first light in a first wavelength band for light treatment and transmit second light in a second wavelength band for white light imaging;
pressing a distal end of the endoscope cap against a living tissue;
concurrently with the pressing, emitting the second light to acquire a white light image of the living tissue based on the second light; and
concurrently with the pressing, irradiating a treatment target of the living tissue with the first light to treat the living tissue.

16. The endoscope cap according to claim 2, wherein
the coating layer is provided on an inner surface of the light-transmissive portion.

17. The endoscope cap according to claim 2, wherein
a distal end of the light-transmissive portion is inclined to intersect a central axis of the light-transmissive portion.

18. The endoscope cap according to claim 17, wherein the coating layer is configured to diffuse light, the coating layer being provided circumferentially on less than an entire circumferential periphery of the light-transmissive portion including on a long side of the light-transmissive portion.

19. The endoscope cap according to claim 2, wherein
the coating layer is configured such that a treatment target is irradiated with the light in the wavelength band for light treatment, and the coating layer is configured to reflect fluorescence from the treatment target excited by the light in the wavelength band for light treatment.
